(19) United States
(12) Patent Application Publication
(10) Pub. No.: US 2007/0135513 A1
(43) Pub. Date: Jun. 14, 2007

(54) Method for producing {n-[1-(s)-carbalkoxy-3-phenylpropyl]-s-alanyl-2s, 3ar, 7as-octahydroindole-2-carboxylic acid} compounds

(76) Inventors: Mirko Pogutter, Schaffhausen (CH); Felix Rudolf, Schaffhausen (CH); Hans-Ulrich Bichsel, Horhausen (CH); Thomas Bader, Zurich (CH)

Correspondence Address: HAMMER & HANF, PC, 3125 SPRINGBANK LANE, SUITE G, CHARLOTTE, NC, 28226, US

(21) Appl. No.: 10/580,638

(22) PCT Filed: Nov. 15, 2004
(86) PCT No.: PCT/CH04/00688

§ 371 Date: Feb. 8, 2007

(30) Foreign Application Priority Data
Nov. 28, 2003 (CH) 2038/03

(51) Int. Cl. A61K 31/405; C07D 209/42

(52) U.S. Cl. 514/419; 548/492

(57) ABSTRACT

A method for preparing optionally substituted {N-[1-(S)-carbalkoxy-3-phenylpropyl]-S-alanyl-2S,3aR,7aS-octahydroindole-2-carboxylic acid} and pharmaceutically acceptable salts thereof, wherein a racemic mixture of optionally substituted trans-octahydroindole-2-carboxylic acid is reacted with the N-carboxyanhydride of {N-[1-(S)-alkoxycarbonyl-3-phenylpropyl]-L-alanine}, which is optionally substituted on the phenyl ring, in a suitable inert solvent, and subsequently the resulting optionally substituted {N-[1-S-carbalkoxy-3-phenylpropyl]-S-alanyl-2S,3aR,7aS-octahydroindole-2-carboxylic acid}, preferably trandolapril, is isolated, and polymorphic forms A and B of trandolapril.

The present invention relates to a method for preparing {N-[1-(S)-carbalkoxy-3-phenylpropyl]-S-alanyl-2S,3aR,7aS-octahydroindole-2-carboxylic acid} compounds and in particular the compound {N-[1-S-carbethoxy-3-phenylpropyl]-S-alanyl-2S,3aR,7aS-octahydroindole-2-carboxylic acid}, which is also known under the name trandolapril. Trandolapril is an active ingredient which, owing to inhibition of angiotensin converting enzyme (ACE), has blood pressure-lowering properties and is employed in particular for the treatment of high blood pressure and heart failure. Trandolapril corresponds to the formula (I):

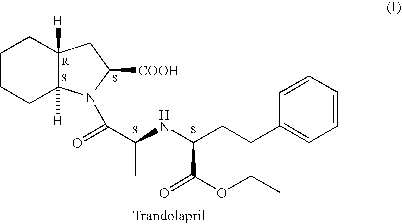

EP 0 084 164 discloses the synthesis of trandolapril by esterifying trans-octahydroindole-2-carboxylic acid with a protective group and subsequently reacting with {N-[1-(S)-ethoxycarbonyl-3-phenylpropyl]-L-alanine in a peptide coupling. The resulting product is then fractionated into the diastereomers by chromatography, after which trandolapril is obtained by eliminating the protective group from the appropriate diastereomer. In this case, the octahydroindole-2-carboxylic acid has the trans configuration and is employed as benzyl or tert-butyl ester in the racemic form or as enantiopure compound in the peptide coupling. EP 0 088 341 and the publication J. Med. Chem. 1987, 30, 992-998 describe analogous syntheses of trandolapril diastereomers. These start from octahydroindole-2-carboxylic ester in the cis configuration and employ for the peptide coupling besides dicyclohexylcarbodiimide or hydroxybenzotriazole also carbonyl-diimidazole. It is particularly disadvantageous in said syntheses that in each case trans-octahydroindole-2-carboxylic acid must be provided with a protective group, and a previous racemate separation of the racemic trans-octahydroindole-2-carboxylic acid employed as coupling component is necessary.

EP 0 215 335 describes a method for preparing {N-[1-(S)-ethoxycarbonyl-3-phenylpropyl]-L-alanyl-L-proline} by reacting the N-carboxyanhydride of {N-[1-(S)-ethoxy-carbonyl-3-phenylpropyl]-L-alanine} with L-proline. It is found in this case that the reaction of N-carboxyanhydrides has no general applicability for controlled and reproducible preparation of heteropeptides and is applicable only to the invention claimed in EP 0 215 335.

The following definitions apply in the present text:
“ECAPPA” means {N-[1-(S)-ethoxycarbonyl-3-phenylpropyl]-L-alanine}.
“NCA” means an N-carboxyanhydride.
“ECAPPA-NCA” means the N-carboxyanhydride of ECAPPA.
“rac.” means “racemic”.
“rac. trans-octahydroindole-2-carboxylic acid” means a racemic mixture of trans-octahydroindole-2-carboxylic acid.

It has now been found that it is possible to prepare trandolapril reproducibly in high yield without interfering side reactions by reacting rac. trans-octa-hydroindole-2-carboxylic acid (i.e. without protective group) with the N-carboxyanhydride of {N-[1-(S)-ethoxycarbonyl-3-phenylpropyl]-L-alanine}, and subsequently to obtain trandolapril from the reaction mixture directly in very pure form by crystallization. Separation of diastereomers by chromatography is unnecessary. In this connection, rac. trans-octahydroindole-2-carboxylic acid specifically means a racemic mixture of (2S,3aR,7aS)-octahydroindole-2-carboxylic acid and (2R,3aS,7aR)-octa-hydroindole-2-carboxylic acid. Analogous statements apply in each case to the claimed substituted compounds.

The present invention provides a simple way of using rac. trans-octahydroindole-2-carboxylic acid (without use of protective groups) and ECAPPA-NCA as starting materials for preparing trandolapril, without the need for a previous racemate separation of rac. trans-octahydroindole-2-carboxylic acid. It is surprising that trandolapril can be obtained directly from the racemate in such pure form by crystallization. Moreover, the reaction according to the invention proceeds without further racemization and allows aqueous workup of the reaction mixture, i.e. the ECAPPA-NCA reaction mixture used in the peptide coupling, to destroy excess reagents such as, for example, triphosgene and byproducts, as is described hereinafter.

The present invention also provides a method permitting separation of diastereomers A1 and B1(see scheme 1 below) by crystallization so that no intermediate purification is necessary until the desired diastereomer is isolated by crystallization. It is possible in this connection for the diastereomers to be separated by crystallization either after salt formation (e.g. as hydrochloride, see method 1 below) or preferably without additional conversion into a salt (see method 2 below). To date, chromatographic methods which are technically difficult to apply have been described for separating corresponding diastereomeric compounds.

Subsequent to the crystallization of the product, elutriation under mild conditions in a suitable medium such as, for example, acetone/water or in acetone is sufficient. The yields of the elutriations are very high and afford the final product in high purity. Overall, the process of the invention is distinguished by technical easy and fast performability.

It has moreover been found that specifically trandolapril crystallizes in two different polymorphic forms and that these different forms also exhibit different properties such as, for example, different bioavailabilities, solubilities and dissolution rates, resulting in appropriate advantages in the production of different administration forms.

The compounds used according to the invention correspond to the following chemical formulae:

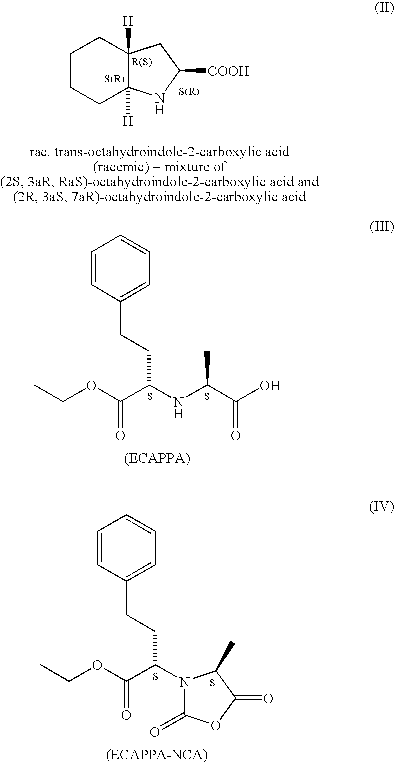

The present invention is defined in the claims. The present invention relates in particular to a method for preparing optionally substituted {N-[1-(S)-carbalkoxy-3-phenylpropyl]-S-alanyl-2S,3aR,7aS-octahydroindole-2-carboxylic acid} and pharmaceutically acceptable salts thereof, which is characterized in that a racemic mixture of optionally substituted trans-octahydroindole-2-carboxylic acid is reacted with the N-carboxyanhydride of {N-[1-(S)-alkoxycarbonyl-3-phenylpropyl]-L-alanine}, which is optionally substituted on the phenyl ring, in a suitable inert solvent, and subsequently the resulting optionally substituted {N-[1-S-carbalkoxy-3-phenylpropyl]-S-alanyl-2S,3aR,7aS-octahydroindole-2-carboxylic acid} is isolated.

The compound is preferably isolated by crystallization. The compound {N-[1-S-carbethoxy-3-phenylpropyl]-S-alanyl-2S,3aR,7aS-octahydroindole-2-carboxylic acid}(trandolapril) is preferably prepared.

The procedure for isolating the compound by crystallization may be such that the resulting diastereomer mixture is converted into a suitable salt, for example in to the hydrochloride, the desired diastereomeric salt is crystallized and then the desired compound, e.g. trandolapril, is liberated therefrom. This method is referred to herein as method 1 (depicted in scheme 1). The compound obtained in this way can subsequently be converted into a suitable salt.

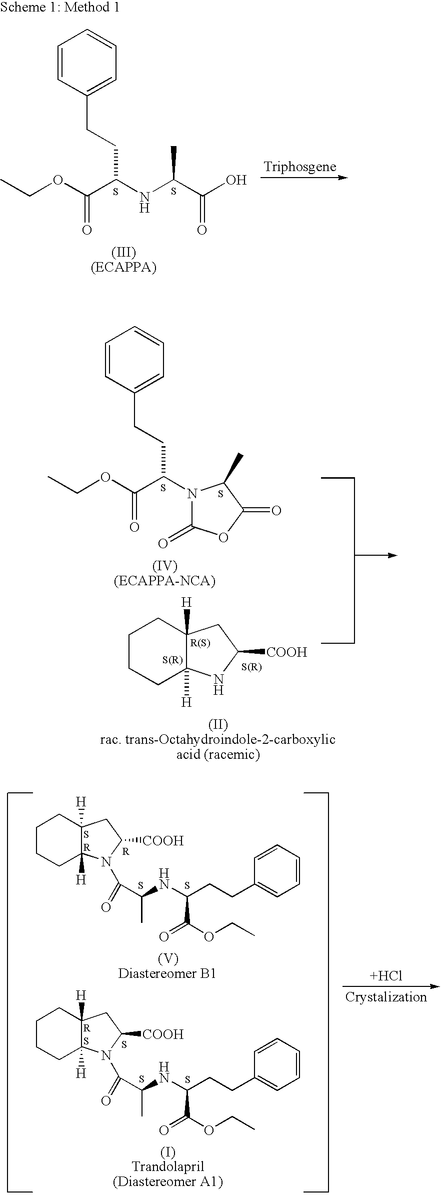

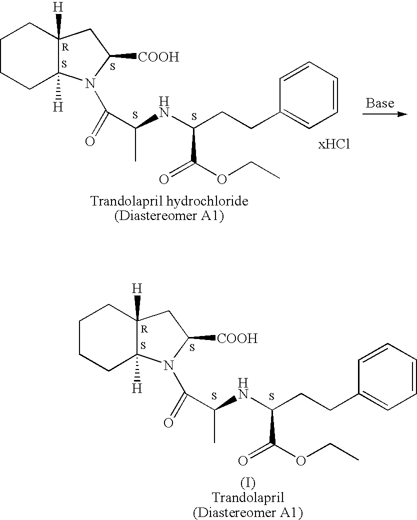

The desired diastereoisomer is preferably crystallized directly from the reaction mixture, i.e. without previous salt formation, so that trandolapril or a derivative of this compound is obtained directly. This preferred method is referred to herein as method 2. The compound prepared in this way can subsequently be converted into a suitable salt. The preparation referred to as method 2 follows scheme 1, but the compound referred to as diastereomer A1is crystallized directly without salt formation.

Optionally substituted trans-octahydroindole-2-carboxylic acid and racemic mixtures thereof are known per se. The unsubstituted carboxylic acid and racemic mixtures thereof is preferably used. The preparation of the N-carboxyanhydride of {N-[1-(S)-ethoxycarbonyl-3-phenylpropyl]-L-alanine} is likewise known.

The preparation of optionally substituted [N-(1-S-carbalkoxy-3-phenylpropyl)-S-alanyl-2S, 3aR,7aS-octahydroindole-2-carboxylic acid] compounds preferably means those compounds in which “carbalkoxy” (identical to “alkoxycarbonyl”) means carbethoxy, carbopropoxy and carbobutoxy, preferably carbethoxy, and the 3-phenylpropyl radical is optionally substituted on the phenyl by methyl, ethyl, propyl or butyl, preferably in the ortho or para position. The 3-phenylpropyl radical is preferably unsubstituted.

Pharmaceutically acceptable salts of this optionally substituted {N-[1-(S)-carbalkoxy-3-phenylpropyl]-S-alanyl-2S,3aR,7aS-octahydroindole-2-carboxylic acid} are in particular those with hydrochloric acid, oxalic acid, tartaric acid, methylsulfonic acid (mesylate), benzenesulfonic acid (besylate), and the other salts described in the literature.

Optionally substituted trans-octahydroindole-2-carboxylic acid and racemic mixtures thereof, and the preparation of {N-[1-(S)-ethoxycarbonyl-3-phenylpropyl]-L-alanine} are known per se. Preparation of the N-carboxyanhydride (NCA) of ECAPPA is likewise known. ECAPPA-NCA is prepared for example by reacting ECAPPA with a carbonyl compound which comprises suitable leaving groups, such as carbonyldiimidazole, trichloromethyl chloroformate, phosgene, diphosgene or triphosgene, preferably with triphosgene.

The method of the invention starts with the preparation of the N-carboxyanhydride in an inert organic solvent at about 0-40° C. This entails heating {N-[1-(S)-alkoxycarbonyl-3-phenylpropyl]-L-alanine}, which is optionally substituted on the phenyl ring, in methylene chloride or another suitable solvent, in the presence of a carbonyl compound which comprises suitable leaving groups, preferably triphosgene, with formation of the NCA. The solvent and the unreacted carbonyl compound are subsequently preferably removed. The remaining product can then be reacted with rac. octahydroindole-2-carboxylic acid to give the diastereomer mixture (A1 and B1, see scheme 1). The desired diastereomer A1, preferably trandolapril, can then be crystallized from the mixture as salt, e.g. as hydrochloride, preferably without conversion into a salt.

Reaction of the NCA of {N-[1-(S)-alkoxycarbonyl-3-phenylpropyl]-L-alanine} with rac. octahydroindole-2-carboxylic acid to give the diastereomer mixture A1 and B1 preferably takes place at a temperature in the range from about −20° C. to room temperature, preferably in the range from about −20° C. to 0° C., with the NCA of {N-[1-(S)-alkoxycarbonyl-3-phenylpropyl]-L-alanine} preferably being added to a suspension of rac. trans-octahydroindole-2-carboxylic acid in a mixed aqueous solvent system. The molar ratio of the NCA, preferably of ECAPPA-NCA, to rac. trans-octahydro-indole-2-carboxylic acid is preferably in the range from 1:1 to 1:1.6, preferably about 1:1.3. Moreover, the acid value (pH) is kept preferably in the basic range, preferably in the range from pH 9 to pH 10, during the reaction, which is achieved by simultaneous addition of an inorganic or organic basically reacting compound.

Examples of such inorganic or organic basically reacting compounds are alkali metal hydroxides, alkali metal carbonates or alkali metal bicarbonates, preferably of sodium or potassium, or secondary or tertiary amines such as, for example, dialkylamines such as dimethylamine, diethylamine, trialkylamines such as trimethylamine, triethylamine, tripropylamine or tributylamine. It is also possible to use for example pyridine or quaternary ammonium hydroxides.

Mixed aqueous solvent systems are preferably mixtures of water and of a water-miscible organic solvent such as, for example, acetone, dioxane or tetrahydrofuran. Acetone is preferred.

After the reaction is complete, the organic solvent is distilled off, resulting initially in an aqueous solution which is then taken up with a water-immiscible organic solvent, for example in an organic ester such as, for example, methyl acetate, ethyl acetate, propyl acetate, preferably ethyl acetate. This entails initially the aqueous and the organic phase being brought with an acid, e.g. by adjusting the aqueous phase with an inorganic acid to an acid value (pH) in the range of pH =4.5-6.0 and subsequent shaking of the two phases, to this acid value, followed by separation from the aqueous phase and concentration of the organic phase. This organic phase now comprises the desired reaction product as diastereomer A1mixed with diastereomer B1, as shown in scheme 1. Selective crystallization of the resulting product, preferably trandolapril, from the organic phase can now be undertaken.

The selective crystallization is preferably carried out at a temperature in the range from −5° C. to +30° C. Since the organic phase comprises the desired reaction product as diastereomer A1 mixed with diastereomer B1, usually in the ratio of about 1:1, it is necessary to separate diastereomer A1 from diastereomer B1. This is surprisingly possible by crystallization.

It has been found that on crystallization of trandolapril, both as hydrochloride (by method 1) and as free compound (by method 2), the water content of the solvent plays a crucial role. Thus, in method 1, a water content of the organic solvent of preferably in the range of 2-4% by weight, preferably of 2.5-3.5% by weight and preferably of about 3% by weight, is used. In this case, the desired diastereomer A1 crystallizes in high purity, while diastereomer B1 remains substantially in solution. The separation at lower water contents is poor or nonexistent. Losses of yield are to be expected with higher water contents.

In method 2, a water content of the organic solvent of preferably in the range of 0.05-4.0% by weight, preferably of 1.5-3.0% by weight, is used. In this case, the desired diastereomer A1 crystallizes in surprisingly high purity, while diastereomer B1 remains substantially in solution. Losses of yield are to be expected with higher water contents, but are not critical.

The solvent preferably used is an organic ester such as, for example, methyl acetate, ethyl acetate, propyl acetate, preferably ethyl acetate.

This crystallization usually results in diastereomer A1 in a purity in the range from 88.0% by weight to 98% by weight, the remaining 2-12% by weight consisting predominantly of ECAPPA and diastereomer B1. Further purification of the product obtained by crystallization can take place by recrystallization or, preferably, by elutriation in an organic solvent or in a mixture of such a solvent with water. Preferred solvents or solvent mixtures are: acetone/water, acetone, acetone/MTBE (methyl tert-butyl ether), ethyl acetate and ethyl acetate/MTBE. The purities of diastereomer A1 obtained with acetone/water at a temperature in the range from 0° C. to room temperature are virtually 100% in this case.

In method 1, diastereomer A1 is isolated by first converting the diastereomer mixture into a suitable salt, and then subjecting it to crystallization. Examples of salts suitable for this purpose are the hydrochloride, sulfate, phosphate, and other salts known per se. The hydrochloride is preferably used. This entails initially the aqueous and the organic phase being brought with an acid, e.g. by adjusting the aqueous phase with an inorganic acid to an acid value (pH) in the range of pH=4.5-6.0 and subsequent shaking of the two phases, to this acid value, followed by separation from the aqueous phase.

The organic phase, preferably ethyl acetate, now comprises the desired reaction product as diastereomer A1 mixed with diastereomer B1. The hydrochloride is prepared by passing HCl gas into the organic phase at 0-20° C., whereupon the hydrochloride is formed. The evaporation of the organic phase results in an oily crude product which is taken up in one of the solvents mentioned, such as acetone with the described water content, and crystallized. Thus, for example, trandolapril hydrochloride is crystallized from acetone/MTBE (methyl tert-butyl ether).

Trandolapril is liberated from the hydrochloride preferably in a mixture of water and a water-miscible organic solvent (e.g. acetone), a pH=4.0-6.0 being adjusted by adding a base. Sodium bicarbonate is preferably used as base. Crystallization of the product may start even during addition of the base at 0-25° C. Further purification of the final product (trandolapril) is possible by recrystallization or, preferably, by elutriation in an organic solvent, possibly mixed with water.

Besides the method described above, the present invention also relates to two novel crystalline forms of trandolapril. It has been found that two different crystalline forms, referred to as form A and form B herein, can be obtained on crystallization of trandolapril.

Crystalline form A is characterized by the following IR and XRD data (tables 1 and 2) and by the ORTEP representation of the corresponding crystal structure analysis (FIG. 1and table 3).

TABLE 1
IR absorption bands of polymorphic form A of trandolapril
Wavelength (cm⁻¹)

```
3446(m)
3280(m)
3063(w)
3028(w)
2994(sh)
2973(w)
2943(m)
2881(w)
2849(w)
1736(s)
1705(sh)
1655(s)
1601(w)
1497(m)
1474(sh)
1457(s)
1434(s)
1397(m)
1367(s)
1356(sh)
1340(w)
1338(w)
1319(m)
1311(m)
1299(w)
1281(m)
1247(m)
1236(m)
1207(sh)
1194(s)
1174(sh)
1109(sh)
1102(m)
1064(m)
1024(m)
  936(m)
  795(m)
  737(m)
  699(m)
```

TABLE 2
XRD data of polymorphic form A of trandolapril

| | Lattice spacing | Relative intensity |
|---|---|---|
| Angle 2 theta (°) | d (Å) | I/Iₘₐₓ (%) |
| 7.34 | 12.07 | 31 |
| 8.88 | 9.99 | 8 |
| 10.66 | 8.33 | 1 |
| 11.66 | 7.63 | 3 |
| 12.3 | 7.24 | 20 |
| 12.54 | 7.1 | 6 |
| 12.88 | 6.92 | 12 |
| 14.58 | 6.12 | 18 |
| 15.66 | 5.71 | 19 |
| 16.44 | 5.45 | 18 |
| 17 | 5.27 | 100 |
| 17.82 | 5.04 | 12 |
| 18.2 | 4.94 | 11 |
| 18.64 | 4.82 | 43 |
| 19.76 | 4.56 | 10 |
| 21.08 | 4.29 | 30 |
| 21.36 | 4.23 | 37 |
| 21.52 | 4.2 | 38 |
| 22.1 | 4.1 | 25 |
| 22.9 | 3.96 | 18 |
| 23.14 | 3.92 | 8 |
| 23.54 | 3.86 | 7 |
| 24.28 | 3.75 | 25 |
| 25.08 | 3.64 | 18 |
| 25.94 | 3.52 | 18 |
| 26.68 | 3.43 | 20 |
| 27.78 | 3.31 | 20 |
| 29.38 | 3.14 | 18 |

Note:
As is known, the intensities may vary owing to texture effects

[image] TABLE 3
Crystal data and structural parameters of trandolapril

| | |
|---|---|
| Identification code | trando |
| Empirical formula | C24H34N2O5 |
| Formula weight | 430.53 |
| Temperature | 160(2) K |
| Wavelength | 0.71073 Å |
| Crystal system, space group | orthorhombic, P2₁2₁2₁ |
| Unit cell dimensions | a = 7.6078(11) Å α = 90 deg. |
| | b = 15.1017(13) Å β = 90 deg. |
| | c = 20.131(3) Å γ = 90 deg. |
| Volume | 2312.8(5) Å³ |
| Z, Calculated density | 4, 1.236 Mg/m³ |
| Absorption coefficient | 0.086 mm⁻¹ |
| F(000) | 928 |
| Crystal size | ca. 0.3 × 0.05 × 0.02 mm |
| Theta range for data collection | 2.70 to 24.85 deg. |
| Limiting indices | 0 ≦ h ≦ 8, 0 ≦ k ≦ 17, 0 ≦ l ≦ 22 |
| Reflections collected/unique | 1829/1829 [R(int) = 0.0000] |
| Completeness to theta = 24.85 | 79.90% |
| Refinement method | Full-matrix least-squares on F² |
| Data/restraints/parameters | 1829/0/283 |
| Goodness-of-fit on F\hat{ }2 | 0.845 |
| Final R indices [I > 2sigma(I)] | R1 = 0.0733, wR2 = 0.1796 |
| R indices (all data) | R1 = 0.1469, wR2 = 0.2405 |
| Absolute structure parameter | −2(5) |
| Largest diff. peak and hole | 0.218 and −0.241 e.Å⁻³ |

The second crystalline form (form B) of trandolapril is characterized by the following IR and XRD data (tables 4 and 5):

TABLE 4
IR absorption bands of polymorphic form B of trandolapril
Wavelength (cm⁻1)

```
3362(m)
3300(sh)
3004(w)
2964(m)
2922(m)
2887(m)
2862(m)
2847(sh)
2492(m)
1954(m)
1740(s)
1721(sh)
1615(s)
1497(m)
1455(s)
1444(sh)
1435(sh)
1377(m)
1362(m)
1345(m)
1297(m)
1288(m)
1281(sh)
1238(sh)
1228(s)
1206(m)
1186(s)
1166(s)
1128(m)
1093(m)
1054(m)
1028(m)
  979(w)
  942(w)
  879(w)
  853(w)
  826(m)
  741(m)
  701(m)
```

TABLE 5
XRD data of polymorphic form B of trandolapril

| | Lattice spacing | Relative intensity |
|---|---|---|
| Angle 2 theta (°) | d (Å) | I/Iₘₐₓ (%) |
| 7.76 | 11.42 | 100 |
| 9.12 | 9.73 | 5 |
| 11.12 | 7.99 | 30 |
| 12.58 | 7.08 | 16 |
| 13.8 | 6.46 | 3 |
| 14.9 | 6.00 | 25 |
| 15.5 | 5.77 | 17 |
| 15.86 | 5.64 | 9 |
| 17.44 | 5.14 | 30 |
| 17.72 | 5.07 | 12 |
| 18.8 | 4.78 | 53 |
| 19.94 | 4.52 | 28 |
| 22.12 | 4.09 | 9 |
| 23.28 | 3.90 | 69 |
| 24.18 | 3.76 | 11 |
| 24.94 | 3.66 | 11 |
| 25.32 | 3.61 | 11 |
| 26.56 | 3.45 | 8 |
| 27.16 | 3.38 | 15 |
| 28.14 | 3.27 | 9 |
| 29.02 | 3.18 | 20 |
| 31.26 | 2.97 | 21 |
| 32.2 | 2.89 | 8 |

Note:
As is known, the intensities may vary owing to texture effects

The stable crystalline form A can be prepared by crystallizing trandolapril from an organic solvent or a mixture of organic solvents (e.g. acetone/cyclohexane), and in this case the water content of the solvent should preferably not exceed 0.2% by weight (<0.2% by weight). In this sense, polymorphic form A is to be referred to as the anhydrous form.

The stable polymorphic form A can be obtained from the less stable form B by elutriation in acetone.

Crystalline form B can be obtained in particular by crystallizing trandolapril from water or mixed aqueous systems at 0-25° C. Polymorphic form B can be prepared specifically in this way by crystallizing trandolapril from methanol/water or acetone/water mixtures at 0-25° C., and form B has a water content in the range of 4-4.4% by weight and may be referred to as monohydrate.

Both forms A and B can be used according to the invention as therapeutic active ingredients and be processed together with a suitable pharmaceutical carrier material to give a medicament. This medicament can be used for treating cardiovascular diseases, specifically for treating high blood pressure and heart failure. Suitable pharmaceutical carrier materials for producing medicaments are known to the skilled worker.

The following examples of the preparation of trandolapril and of polymorphic forms A and B of trandolapril illustrate the present invention without restricting its scope and its application.

The XRD spectra were recorded with a Philips ADP1700 powder diffractometer with Cu radiation of K_{α1}=0.15406 nm and K_{α2}=0.15444 and a voltage of 40 kV.

### EXAMPLE 1

### Preparation of Trandolapril by Method 1

a. Preparation of ECAPPA-NCA:

61.45 g of ECAPPA are dissolved in 580 g of methylene chloride at 20-30° C. and, at this internal temperature, a solution of 62.32 g of triphosgene in 212 g of methylene chloride is added. The mixture is then heated under reflux for 14-16 hours. After conversion is complete, the mixture is concentrated in vacuo (600 to <50 mbar), and the resulting viscous yellow oil is taken up in 126.8 g of acetone at 10-20° C. The solution is cooled to 0-5° C. and added dropwise to a suspension of 33.6 g of sodium bicarbonate in 82 g of water at 0-8° C. After addition is complete, the two-phase NCA suspension is stirred at 0-5° C. for 30-90 minutes.

b. Coupling of ECAPPA-NCA to rac. trans-octahydroindole-2-carboxylic acid:

A suspension of 33.85 g of rac. trans-octahydroindole-2-carboxylic acid in 140 g of acetone and 216 g of water is cooled to 0-5° C., and 1.5 g of triethylamine is added (pH=10.6). The ECAPPA-NCA suspension prepared above under a. is added dropwise to this suspension at 0-10° C., keeping the pH in the range 9.0-10.0 by the simultaneous addition of a total of 49.4 g of triethylamine. The reaction mixture is then stirred at 0-5° C. for 1 hour and at 20-25° C. for 1 hour and subsequently filtered, and the filter cake is washed with 40 g of acetone. The acetone is almost completely removed from the filtrate under 200 mbar.

c. Preparation of Trandolapril Hydrochloride and Separation of the Diastereomers:

The concentrated filtrate from section b. is taken up in 600 g of ethyl acetate at 20-25° C. and adjusted to a pH=5.0-5.5 with a solution of 20.3 g of concentrated hydrochloric acid in 20.3 g of water at 15-20° C. The organic phase is separated off, dried over sodium sulfate and cooled to 0-5° C. In total, 29.17 g of HCl gas is slowly passed into this solution. The solvent is then removed in vacuo, the resulting clear oil is taken up in 320 g of acetone, and the solution is heated to 55° C. 640 g of MTBE and then a little trandolapril hydrochloride (for seeding) are added to the hot solution. This results in a suspension which is stirred at 55° C. for 10 minutes, at 20-25° C. for 90 minutes and at 0-5° C. for 60 minutes. The suspension is filtered with suction and the solid is dried in vacuo (yield: 30.01 g of trandolapril hydrochloride).

d. Liberation of trandolapril from the hydrochloride:

A solution of 4.72 g of sodium bicarbonate in 89.05 g of water is added to a solution of 30.01 g of trandolapril hydrochloride in 240 g of water and 60 g of acetone at 20-25° C. The pH of the solution is then about 4.5. The suspension resulting therefrom is stirred at 20-25° C. for 1 hour and then at 0-5° C. for 1 hour and subsequently filtered. The solid is washed with water and dried in vacuo (yield: 23.04 g).

e. Purification of Trandolapril:

23.04 g of trandolapril are stirred in 147 g of acetone initially at 20-25° C. for 20 minutes and then at 0-5° C. for 1 hour. After filtration, the solid is washed with acetone and dried in vacuo (yield: 21.93 g; HPLC purity: ≧99.9%).

### EXAMPLE 2

### Preparation of Trandolapril by Method 2

a. Preparation of ECAPPA-NCA:

30.73 g of ECAPPA are dissolved in 264 g of methylene chloride at 20-30° C. and, at this internal temperature, a solution of 31.16 g of triphosgene in 132 g of methylene chloride is added. The mixture is then heated under reflux for 14-16 hours. After conversion is complete, the mixture is concentrated in vacuo (600 to 100 mbar), and the resulting viscous yellow oil is taken up in 64 g of acetone at 10-20° C. The solution is cooled to 0-5° C. and added dropwise to a suspension of 16.8 g of sodium bicarbonate in 41 g of water at 0-10° C. After addition is complete, the two-phase NCA suspension is stirred at 0-5° C. for 30-90 minutes.

b. Coupling of ECAPPA-NCA to rac. trans-octahydroindole-2-carboxylic acid:

A suspension of 16.92 g of rac. trans-octahydroindole-2-carboxylic acid in 56 g of acetone and 86 g of water is stirred at 20-25° C.° C., and 0.5 g of triethylamine is added (pH=9.65). The ECAPPA-NCA suspension prepared under a. (cooled to 0-5° C.) is added dropwise to this suspension at 20-25° C., keeping the pH in the range 9.0-9.7 by simultaneous addition of a total of 28.16 g of triethylamine. The reaction mixture is then stirred at 20-25° C. for 2 hours and subsequently filtered, and the filter cake is washed with 20 g of acetone. The acetone is almost completely removed from the filtrate under 200-100 mbar.

c. Separation of the Diastereomers and Isolation of Trandolapril:

The concentrated filtrate from section b. is taken up in 240 g of ethyl acetate at 20-25° C. and adjusted to a pH=5.0-5.5 with a solution of 10.15 g of concentrated hydrochloric acid in 10.5 g of water at 20-25° C. After phase separation, 50-70 g of ethyl acetate are distilled out of the organic phase under 120-160 mbar. The organic phase is then seeded with a little trandolapril and cooled to 0-5° C. The suspension is stirred at 0-5° C. for 2-3 hours and filtered, and the solid is washed with ethyl acetate. Drying in vacuo results in 17.23 g of solid.

d. First Purification of Trandolapril in Water/acetone:

17.23 g of trandolapril crude product from section c. are stirred in 80 g of water and 63.3 g of acetone initially at 20-25° C. for 1 hour and then at 0-5° C. for 1 hour. After filtration, the solid is washed with acetone and dried in vacuo (yield: 13.97 g: HPLC purity:>99.9%).

e. Second Purification of Trandolapril in Acetone:

13.97 g of trandolapril from section d. are stirred in 81.3 g of acetone initially at 20-25° C. for 20 minutes and then at 0-5° C. for 1-1.5 hours. After filtration, the solid is washed with acetone and dried in vacuo (yield: 13.51 g; HPLC purity:>99.9%).

### EXAMPLE 3

### Preparation of Polymorphic Form A of Trandolapril

A solution of 1.00 9 of trandolapril in 60 ml of acetone is added at 0-5° C. to 180 ml of cyclohexane. After crystallization starts, the suspension is stirred at 0-5° C. for 1 hour and then filtered. The moist product is dried in vacuo at 35° C. Form A is obtained in a yield of 0.65 g).

### EXAMPLE 4

### Preparation of Polymorphic Form A of Trandolapril

Polymorphic form B of trandolapril is slurried in acetone as described in example 1, section e) and in example 2, section e), whereupon form B is completely converted into polymorphic form A.

### EXAMPLE 5

### Preparation of Polymorphic Form B of Trandolapril

1.00 g of trandolapril is dissolved in 8 ml of methanol. The solution is then added to 40 ml of water, which contains a little trandolapril of form B, at 0-5° C. After the crystallization starts, the suspension is stirred at 0-5° C. for 2-3 hours and then filtered. The product is dried in vacuo at 40° C. for 12 hours. Trandolapril in polymorphic form B is obtained (yield: 0.93 g).

### EXAMPLE 6

### Preparation of Polymorphic Form B of Trandolapril

A solution of 1.00 g of trandolapril in 60 ml of acetone is added at 0-5° C. to 300 ml of water to which a little trandolapril of form B has previously been added. The solution is stirred at this internal temperature until crystallization starts (5-6 hours) and is stored in a refrigerator overnight. The suspension is then filtered and the solid is dried in vacuo at 40° C. Trandolapril is obtained in polymorphic form B in a yield of 0.29 g.

1. A method for preparing optionally substituted {N-[1-(S)-carbalkoxy-3-phenylpropyl]-S-alanyl-2S,3aR,7aS-octahydroindole-2-carboxylic acid} and pharmaceutically acceptable salts thereof, characterized in that a racemic mixture of optionally substituted trans-octahydroindole-2-carboxylic acid is reacted with the N-carboxyanhydride of {N-[1-(S)-alkoxycarbonyl-3-phenylpropyl]-L-alanine}, which is optionally substituted on the phenyl ring, in a suitable inert solvent, and subsequently the resulting optionally substituted {N-[1-S-carbalkoxy-3-phenylpropyl]-S-alanyl-2S, 3aR,7aS-octahydroindole-2-carboxylic acid} is isolated.

2. The method as claimed in claim 1, characterized in that the compound is isolated by crystallization.

3. The method as claimed in claim 1, characterized in that the compound {N-[1-S-carbethoxy-3-phenylpropyl]-S-alanyl-2S, 3aR,7aS-octahydroindole-2-carboxylic acid}(trandolapril) is prepared.

4. The method as claimed in claim 2 characterized in that the resulting diastereomer mixture is converted into a suitable salt, preferably the hydrochloride, sulfate or phosphate, preferably into the hydrochloride, the desired diastereomer salt is crystallized and then the desired compound, preferably, for example, trandolapril, is liberated therefrom, and the compound obtained in this way is subsequently converted where appropriate into a suitable salt.

5. The method as claimed in claim 2, characterized in that desired diastereomer, preferably trandolapril, is crystallized directly from the reaction mixture and, where appropriate, the compound is subsequently converted into a suitable salt.

6. The method as claimed in claim 1, characterized in that optionally substituted [N-1-S-carbalkoxy-3-phenylpropyl)-S-alanyl-2S,3aR,7aS-octahydroindole-2-carboxylic acid] compounds in which “carbalkoxy” means carbethoxy, carbopropoxy or carbobutoxy, preferably carbethoxy, and the 3-phenylpropyl radical is optionally substituted on the phenyl by methyl, ethyl, propyl or butyl, preferably in the ortho or para position, and is preferably unsubstituted, are prepared.

7. The method as claimed in claim 1, characterized in that a pharmaceutically acceptable salt is prepared, preferably a salt with hydrochloric acid, oxalic acid, tartaric acid, methylsulfonic acid, benzenesulfonic acid.

8. The method as claimed in claim 1, characterized in that the reaction of the NCA of {N- [1-(S) -alkoxycarbonyl-3-phenylpropyl]-L-alanine} with rac. octahydroindole-2-carboxylic acid is carried out at a temperature in the range from about −20 ° C. to room temperature, with the NCA of {N- [1-(S) -alkoxycarbonyl-3-phenylpropyl]-L-alanine} preferably being added to a suspension of rac. trans-octahydroindole-2-carboxylic acid in a mixed aqueous solvent system.

9. The method as claimed in claim 8, characterized in that the molar ratio of the NCA to rac. trans-octahydroindole-2-carboxylic acid is in the range from 1:1 to 1:1.6, and the acid value (pH) is kept in the basic range, during the reaction.

10. The method as claimed in claim 8, characterized in that mixtures of water and of a water-miscible organic solvent, preferably acetone, dioxane or tetrahydrofuran, preferably acetone, is used as mixed aqueous solvent system.

11. The method as claimed in claim 1, characterized in that the crystallization is carried out at a temperature in the range from −5° C. to +30° C., the water content of the organic solvent during the crystallization of the salt being in the range of 2-4% by weight, and the water content of the organic solvent during the crystallization of diastereomer A1 being in the range of 0.05-4.0% by weight.

12. The method as claimed in claim 11, characterized in that an organic ester, preferably methyl acetate, ethyl acetate, propyl acetate, is used as organic solvent.

13. The method as claimed in claim 1, characterized in that the product obtained by crystallization is purified by recrystallization or by elutriation in an organic solvent or in a mixture of such a solvent with water, preferably in acetone/water, acetone, acetone/MTBE, ethyl acetate or ethyl acetate/MTBE.

14. The crystalline polymorphic, substantially anhydrous, form A of trandolapril characterized by the XRD data listed in table 2.

15. The crystalline polymorphic hydrous form B of trandolapril characterized by the XRD data listed in table 5 and by the fact that this form B has a water content in the range of 4-4.4% by weight.

16. A method for preparing polymorphic form A as claimed in claim 14, characterized in that trandolapril is crystallized from an organic solvent or a mixture of organic solvents.

17. The method as claimed in claim 16, characterized in that the water content of the solvent does not exceed 0.2% by weight (<0.2% by weight).

18. A method for preparing polymorphic form B as claimed in claim 15, characterized in that trandolapril is crystallized from water or mixed aqueous systems at 0-25° C.

19. The use of form A as claimed in claim 14 as therapeutic active ingredients, preferably for treating cardiovascular diseases, preferably for treating high blood pressure and heart failure.

20. The use of form B as claimed in claim 15 as therapeutic active ingredients, preferably for treating cardiovascular diseases, preferably for treating high blood pressure and heart failure.
